(19) United States
(12) Patent Application Publication
(10) Pub. No.: US 2022/0160276 A1
(43) Pub. Date: May 26, 2022

(54) CONCENTRATION DEGREE MEASUREMENT DEVICE, CONCENTRATION DEGREE MEASUREMENT METHOD, AND RECORDING MEDIUM

(71) Applicant: Panasonic Intellectual Property Management Co., Ltd., Osaka (JP)

(72) Inventors: Kaiyu SUZUKI, Osaka (JP); Mototaka YOSHIOKA, Osaka (JP); Souksakhone BOUNYONG, Nara (JP); Kunihiro IMAMURA, Osaka (JP)

(21) Appl. No.: 17/441,449

(22) PCT Filed: Mar. 19, 2020
(86) PCT No.: PCT/JP2020/012292

§ 371 Date: Sep. 21, 2021

(30) Foreign Application Priority Data
Mar. 29, 2019 (JP) 2019-065297

(51) Int. Cl. A61B 5/16; G06T 7/70; G06T 7/20; G06V 40/20; G08B 21/06; A61M 21/00

(52) U.S. Cl. CPC A61B 5/168; G06T 7/70; G06T 7/20; G06V 40/20; A61M 2021/0066; A61M 21/00; G06T 2207/30201; A61M 2021/0083; G08B 21/06

(57) ABSTRACT

A concentration degree measurement device includes: a concentration degree obtainer that obtains a degree of concentration of a person; a drowsiness degree obtainer that obtains a degree of drowsiness of the person; a corrector that corrects the degree of concentration, based on the degree of drowsiness; an outputter that outputs a corrected degree of concentration indicating the degree of concentration corrected by the corrector; a determiner that determines a factor for the corrected degree of concentration, based on the degree of drowsiness and the corrected degree of concentration; and a controller that controls an electronic device, based on a result of the determination by the determiner, the electronic device inducing the person to wake up or concentrate.

### CROSS-REFERENCE OF RELATED APPLICATIONS

This application is the U.S. National Phase under 35 U.S.C. § 371 of International Patent Application No. PCT/JP2020/012292, filed on Mar. 19, 2020, which in turn claims the benefit of Japanese Application No. 2019-065297, filed on Mar. 29, 2019, the entire disclosures of which Applications are incorporated by reference herein.

### TECHNICAL FIELD

The present disclosure relates to a concentration degree measurement device, a concentration degree measurement method, and a recording medium.

### BACKGROUND ART

Conventionally, information processing devices that calculate a degree of concentration of a person have been known. For example, the information processing device disclosed by Patent Literature (PTL) 1 calculates a degree of concentration of a person, based on a change in a facial orientation of the person.

### CITATION LIST

### Patent Literature

[PTL 1] Japanese Unexamined Patent Application Publication No. 2014-120137

### SUMMARY OF INVENTION

### Technical Problem

In the conventional information processing devices, when a person is looking at one target object concentratedly, a degree of concentration of the person will be calculated as a high value. This, however, does not necessarily mean that a degree of concentration of the person looking at the one target object is high. For example, when a degree of drowsiness of a person performing a mental task such as study or office work is high, the conventional information processing devices calculate a high degree of concentration even though the person is not really in an attentive state because a change in a facial orientation etc. occurs less frequently. As just descried, the problem with the conventional information processing devices is that the accuracy of a degree of concentration to be calculated is low.

In view of the above, the present disclosure provides a concentration degree measurement device etc. capable of measuring a degree of concentration with high accuracy.

### Solution to Problem

In order to solve the above problem, a concentration degree measurement device according to one aspect of the present disclosure includes: a concentration degree obtainer that obtains a degree of concentration of a person; a drowsiness degree obtainer that obtains a degree of drowsiness of the person; a corrector that corrects the degree of concentration, based on the degree of drowsiness; an outputter that outputs a corrected degree of concentration indicating the degree of concentration corrected by the corrector; a determiner that determines a factor for the corrected degree of concentration, based on the degree of drowsiness and the corrected degree of concentration; and a controller that controls an electronic device, based on a result of the determination by the determiner, the electronic device inducing the person to wake up or concentrate.

Moreover, a concentration degree measurement method according to one aspect of the present disclosure includes: obtaining a degree of concentration of a person; obtaining a degree of drowsiness of the person; correcting the degree of concentration, based on the degree of drowsiness; outputting a corrected degree of concentration indicating the degree of concentration corrected in the correcting; determining a factor for the corrected degree of concentration, based on the degree of drowsiness and the corrected degree of concentration; and controlling an electronic device, based on a result of the determination in the determining, the electronic device inducing the person to wake up or concentrate.

Furthermore, one aspect of the present disclosure can be realized as a program causing a computer to execute the above concentration degree measurement method. Alternatively, one aspect of the present disclosure can be realized as a non-transitory computer-readable recording medium having the program recorded thereon.

### Advantageous Effects of Invention

A concentration degree measurement device etc. according to one aspect of the present disclosure is capable of measuring a degree of concentration with high accuracy.

### BRIEF DESCRIPTION OF DRAWINGS

FIG. 1 is a schematic diagram illustrating a state of a user whose degree of concentration is measured according to Embodiment 1.

FIG. 2A is a diagram schematically illustrating a line of sight of the user.

FIG. 2B is a diagram schematically illustrating a line of sight of the user.

FIG. 2C is a diagram schematically illustrating a line of sight of the user.

FIG. 3 is a block diagram illustrating a configuration of a concentration degree measurement device according to Embodiment 1.

FIG. 4 is a flow chart illustrating a process performed by the concentration degree measurement device according to Embodiment 1.

FIG. 5 is a block diagram illustrating a configuration of a concentration degree measurement device according to Embodiment 2.

FIG. 6 is a flow chart illustrating a process performed by the concentration degree measurement device according to Embodiment 2.

### DESCRIPTION OF EMBODIMENTS

[Overview of the present disclosure]

In order to solve the above problem, a concentration degree measurement device according to one aspect of the present disclosure includes: a concentration degree obtainer that obtains a degree of concentration of a person; a drowsiness degree obtainer that obtains a degree of drowsiness of the person; a corrector that corrects the degree of concentration, based on the degree of drowsiness; and an outputter that outputs a corrected degree of concentration indicating the degree of concentration corrected by the corrector.

With such a configuration, the concentration degree measurement device is capable of correcting a degree of concentration of a person, based on a degree of drowsiness of the person. For this reason, calculating the degree of concentration to be high is suppressed even though the degree of drowsiness is high and the degree of concentration is low. As stated above, the concentration degree measurement device according to one aspect of the present disclosure is capable of measuring a degree of concentration with high accuracy.

Moreover, for example, the corrector makes the correction so that the degree of concentration decreases with an increase in the degree of drowsiness.

A high degree of drowsiness of a person can be considered equivalent to a low degree of concentration of the person. Accordingly, by correcting a degree of concentration of the person to be lower as a degree of drowsiness of the person is higher, the degree of concentration is measured with high accuracy.

Moreover, for example, the corrector makes the correction so that the corrected degree of concentration is lower than the degree of concentration, based on the degree of drowsiness.

A degree of concentration of a person is considered to decrease with an increase in a degree of drowsiness of the person. For this reason, by correcting the degree of concentration to be lower based on the degree of drowsiness, the degree of concentration is measured with ease and high accuracy.

Moreover, for example, the concentration degree measurement device further includes a controller that controls an electronic device, based on the degree of drowsiness and the corrected degree of concentration, the electronic device inducing the person to wake up or concentrate.

With such a configuration, since the degree of concentration and the degree of drowsiness are measured with high accuracy, the controller is capable of inducing the person to wake up or concentrate at an appropriate timing.

Moreover, for example, the concentration degree measurement device further includes a determiner that determines whether the corrected degree of concentration is lower than a first threshold value, and determines, when the determiner determines that the corrected degree of concentration is lower than the first threshold value, a factor that has caused the corrected degree of concentration to be lower than the first threshold value. The controller controls the electronic device, based on a result of the determination by the determiner.

Such a configuration calculates how much the degree of drowsiness contributes to the factor that has caused the degree of concentration to be lower. Accordingly, the controller is capable of inducing the person to wake up or concentration at a more appropriate timing.

Moreover, for example, when the determiner determines that a proportion of drowsiness in the factor is higher than a second threshold value, the controller causes the electronic device to induce the person to wake up; and when the determiner determines that the proportion of the drowsiness in the factor is not higher than the second threshold value, the controller causes the electronic device to induce the person to concentrate.

With such a configuration, the determiner appropriately determines whether to induce the person to wake up or concentrate. For this reason, the controller is capable of more appropriately inducing the person to wake up or concentrate.

Moreover, for example, the controller performs inverse control on the electronic device between when the electronic device induces the person to wake up and when the electronic device induces the person to concentrate.

For example, an increase in environmental temperature induces a person to concentrate. In contrast, a decrease in environmental temperature induces a person to wake up. Furthermore, for example, an increase in environmental noise induces a person to wake up. In contrast, a decrease in environmental noise induces a person to concentrate. As stated above, by switching control modes, it is possible to cause one electronic device such as air-conditioning equipment and audio equipment to induce the person to wake up or concentrate. Accordingly, by the controller performing inverse control on the electronic device between when the electronic device induces the person to wake up and when the electronic device induces the person to concentrate, it is possible to induce the person to wake up or concentrate using the simple configuration.

Moreover, for example, the corrector changes a manner of correcting the degree of concentration, based on environmental information indicating an environmental state of the person.

For example, when an environmental temperature is relatively high, a change in a degree of concentration and a degree of drowsiness of a person has a tendency depending on an environmental condition of the person, such as a tendency of the degree of concentration and the degree of drowsiness to increase. For this reason, by correcting the degree of concentration, based on the environmental condition of the person, the concentration degree measurement device according to the present disclosure is capable of measuring the degree of concentration with higher accuracy.

Moreover, for example, the concentration degree obtainer obtains the degree of concentration by calculating the degree of concentration, based on at least one of a line of sight or a facial orientation of the person in an image obtained from an image capturer, and the drowsiness degree obtainer obtains the degree of drowsiness by calculating the degree of drowsiness, based on blinking by the person in the image.

Such a configuration makes it possible to obtain a degree of drowsiness and a degree of concentration of a person from the same image. Accordingly, the degree of concentration and the degree of drowsiness are calculated using the simple configuration.

Moreover, for example, the concentration degree obtainer obtains the degree of concentration by calculating, from images obtained from the image capturer and showing the person and a predetermined target object, an amount of temporal change in the at least one of the line of sight or the facial orientation toward the predetermined target object.

For example, when a person is performing a task such as learning materials displayed by a display device, a predetermined target object, while looking at the task, a degree of concentration of the person is determined to be higher as a time for which the person is looking at the display device is longer. For this reason, with such a configuration, the degree of concentration is measured with higher accuracy.

Moreover, for example, the corrector changes a correction value based on the degree of drowsiness, based on a position of the predetermined target object, the correction value being used to correct the degree of concentration.

For example, a degree of drowsiness of a person is calculated based on a time required for one blinking by the person. Accordingly, for example, when the person is performing a task such as learning materials displayed by a display device, a predetermined target object, while looking at the task, an amount of opening and closing by the eyelids captured by the image capturer changes depending on a positional relationship between the display device, the image capturer, and the person. Therefore, such a configuration reduces deterioration of accuracy of measuring the degree of concentration due to the positional relationship between the predetermined target object, the image capturer, and the person.

A concentration degree measurement method according to one aspect of the present disclosure includes: obtaining a degree of concentration of a person; obtaining a degree of drowsiness of the person; correcting the degree of concentration, based on the degree of drowsiness; and outputting a corrected degree of concentration indicating the degree of concentration corrected in the correcting.

Such a method makes it possible to measure a degree of concentration of a person, based on a degree of drowsiness of the person. For this reason, calculating the degree of concentration to be high is suppressed even though the degree of drowsiness is high and the degree of concentration is low. As stated above, the concentration degree measurement method according to one aspect of the present disclosure makes it possible to measure a degree of concentration with high accuracy.

Moreover, one aspect of the present disclosure can be realized as a program causing a computer to execute the above concentration degree measurement method. Alternatively, one aspect of the present disclosure can be realized as a non-transitory computer-readable recording medium having the program recorded thereon.

Hereinafter, embodiments will be described in detail with reference to the drawings.

It should be noted that each of the embodiments described below shows a generic or specific example. The numerical values, shapes, materials, constituent elements, the arrangement and connection of the constituent elements, steps, the order of the steps, etc. indicated in the following embodiments are mere examples, and thus are not intended to limit the present disclosure. Moreover, among the constituent elements described in the following embodiments, those not recited in any one of the independent claims are described as optional constituent elements.

Furthermore, the respective figures are schematic diagrams and are not necessarily precise illustrations. Accordingly, for example, the scales etc. in the respective figures are not necessarily uniform. Moreover, in the drawings, substantially same elements are assigned the same reference signs, and overlapping description is omitted or simplified.

Furthermore, although the expressions “higher than or equal to a threshold value,” “lower than or equal to a threshold value,” etc. are included in the following description, they are not used in a strict sense. For example, the expression “higher than or equal to a threshold value” may mean higher than a reference value. In addition, when the expressions “higher than or equal to a threshold value” and “lower than a threshold value” are used in contrast with each other, they may mean being distinguished with reference to the reference value and mean “higher than a threshold value” and “lower than or equal to a threshold value,” respectively.

### Embodiment 1

[Overview]

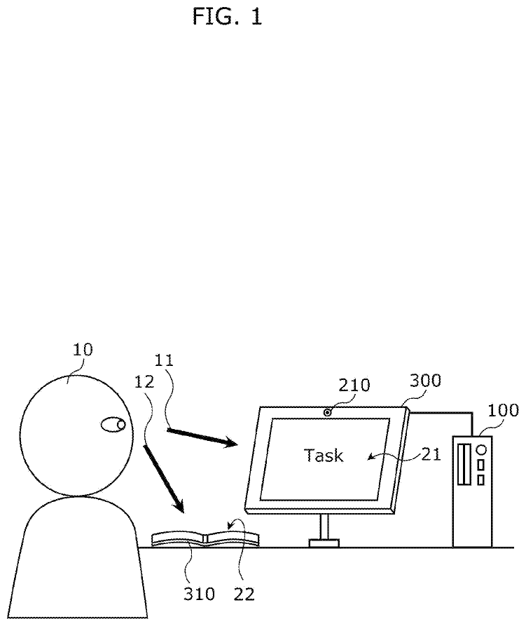

First, the following describes a configuration of a concentration degree measurement device according to Embodiment 1 with reference to FIG. 1 to FIG. 3.

FIG. 1 is a schematic diagram illustrating a state of user 10 whose degree of concentration is measured according to Embodiment 1.

Concentration degree measurement device 100 according to Embodiment 1 measures a degree of concentration of user 10. A degree of concentration is an indicator of how much user 10 is concentrating on work. A degree of concentration is represented by a value in a predetermined range such as 0 to 1, 0 to 10, or 0 to 100. For example, a higher degree of concentration (e.g., a higher value representing a degree of concentration) indicates that user 10 is concentrating more on work, and a lower degree of concentration (a lower value representing a degree of concentration) indicates that user 10 is concentrating less on work.

It should be noted that a value range for a degree of concentration is not particularly limited.

Work (a task) is mental work such as study, learning, reading, or office work, but the present embodiment is not limited to this example. Work may be work using hands and feet such as driving, machine operation, or production of goods. Alternatively, work may be related to movie, music, or art appreciation or sports viewing, or leisure such as a game or sports.

In an example shown by FIG. 1, user 10 is learning using task 21 that is learning content displayed by display device 300. Task 21 is, for example, educational content displayed on a display of display device 300. Moreover, user 10 is learning further using task 22 written in book 310 for learning such as a textbook, a reference book, or a notebook. When user 10 is concentrating on learning (i.e., a degree of concentration is high), a line of sight and a facial orientation of user 10 are directed toward a predetermined target object (display device 300 or book 310 in the present embodiment) on which task 21 is displayed or task 22 is written.

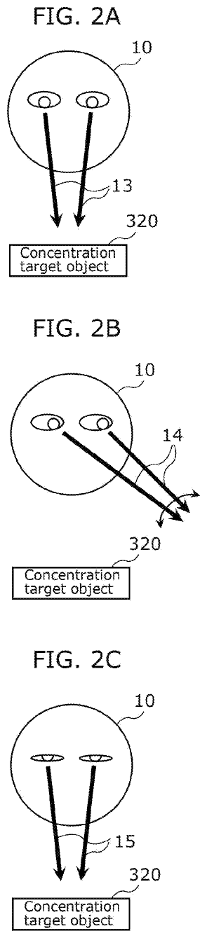

FIG. 2A to FIG. 2C each are a diagram schematically illustrating a line of sight of user 10. Specifically, FIG. 2A is a diagram schematically illustrating that a degree of concentration of user 10 is high. Moreover, FIG. 2B is a diagram schematically illustrating that a degree of concentration and a degree of drowsiness of user 10 are low. Furthermore, FIG. 2C is a diagram schematically illustrating that a degree of concentration and a degree of drowsiness of user 10 are low and high, respectively.

As shown by arrows 13 in FIG. 2A, when the degree of concentration of user 10 is high, the line of sight of user 10 is directed toward concentration target object 320 on which a task is displayed, and a shift amount of the line of sight of user 10 (i.e., a movement amount of the line of sight) within a predetermined time is small.

In contrast, as shown by arrows 14 in FIG. 2B, when the degree of concentration of user 10 is low, the line of sight of user 10 is directed toward something other than concentration target object 320. Moreover, when the degree of concentration of user 10 is low, the shift amount of the line of sight of user 10 is large compared to a state in which the degree of concentration of user 10 is high.

As stated above, a degree of concentration of user 10 can be determined based on the length of a time for which a line of sight of user 10 is directed toward concentration target object 320 and/or a shift amount of the line of sight of user 10.

Here, as shown by FIG. 2C, user 10 is assumed to be drowsy (i.e., a degree of drowsiness is high). At this time, as shown by arrows 15, the line of sight of user 10 may has a small shift amount while directed toward concentration target object 320.

In this case, a conventional information processing device calculates a high value as a degree of concentration of user 10 even though user 10 is not concentrating on a task because user 10 is drowsy. In other words, the conventional information processing device determines the degree of concentration to be high even though user 10 is not concentrating on learning.

On the other hand, concentration degree measurement device 100 according to the present embodiment is capable of, for example, reflecting the lack of concentration on learning on a degree of concentration (a provisional degree of concentration) calculated by the conventional information processing device, by correcting the degree of concentration using a degree of drowsiness of user 10. Specifically, concentration degree measurement device 100 corrects the degree of concentration to a small value when the degree of drowsiness of user 10 is high. Accordingly, concentration degree measurement device 100 is capable of measuring a degree of concentration with high accuracy.

### [Configuration]

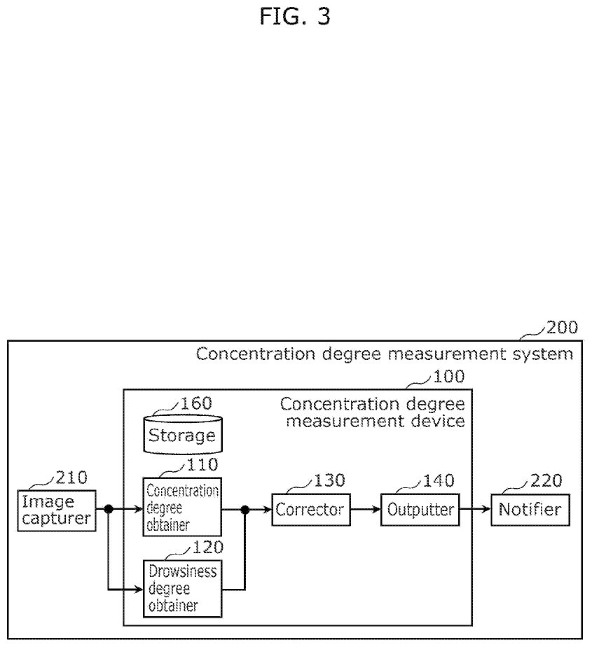

Next, the following describes a specific configuration of concentration degree measurement device 100 with reference to FIG. 1 and FIG. 3.

FIG. 3 is a block diagram illustrating a characteristic functional configuration of concentration degree measurement device 100 according to Embodiment 1. It should be noted that FIG. 3 shows a characteristic functional configuration of concentration degree measurement system 200 including a peripheral configuration of concentration degree measurement device 100.

Concentration degree measurement system 200 causes concentration degree measurement device 100 to measure a degree of concentration of a person (e.g., user 10) using an image captured by image capturer 210 and to notify the person of the degree of concentration. Concentration degree measurement system 200 includes image capturer 210, concentration degree measurement device 100, and notifier 220.

Concentration degree measurement device 100 measures a degree of concentration of user 10. Concentration degree measurement device 100 includes concentration degree obtainer 110, drowsiness degree obtainer 120, corrector 130, outputter 140, and storage 160.

Concentration degree obtainer 110 obtains a degree of concentration (a provisional degree of concentration) of user 10. For example, concentration degree obtainer 110 performs an operation using an image (a captured image) obtained from image capturer 210 to obtain a provisional degree of concentration of user 10. Concentration degree obtainer 110 may calculate a provisional degree of concentration using a conventionally known method of calculating a degree of concentration. For example, concentration degree obtainer 110 calculates a provisional degree of concentration, based on a movement amount of user 10 within a predetermined time. More specifically, concentration degree obtainer 110 calculates a provisional degree of concentration having a high value, assuming that user 10 is concentrating as user 10 moves less within the predetermined time. Concentration degree obtainer 110 calculates a provisional degree of concentration having a low value, assuming that user 10 is not concentrating as user 10 moves intensively within the predetermined time.

In the present embodiment, concentration degree obtainer 110 obtains a degree of concentration of user 10 by calculating the degree of concentration, based on at least one of the line of sight or the facial orientation of user 10 included in an image obtained from image capturer 210. More specifically, concentration degree obtainer 110 obtains the degree of concentration by calculating, from images obtained from image capturer 210 and including user 10 and a predetermined target object (e.g., display device 300 that displays task 21, book 310 in which task 22 is written), an amount of temporal change in at least one of the line of sight or the facial orientation of user 10 toward the predetermined target object (i.e., a shift amount of at least one of the line of sight or the facial orientation within a predetermined time).

As stated above, concentration degree obtainer 110 measures (calculates) at least one of the line of sight or facial orientation of user 10, based on the image obtained from image capturer 210. Specifically, of concentration degree obtainer 110 measures at least one of the line of sight or the facial orientation by performing image processing such as outline extraction on the image.

A line of sight is a direction in which user 10 is looking. In a specific example, a line of sight is represented by arrows 11 and 12 shown by FIG. 1. For example, concentration degree obtainer 110 extracts an iris region from an image and measures a line of sight, based on the shape and center position of the iris extracted.

A facial orientation is an orientation of the face of user 10. Specifically, a facial orientation is represented by the front direction of the face of user 10. For example, concentration degree obtainer 110 detects a face in an image and measures a facial orientation by extracting feature points of the eyes, the mouth, etc.

It should be noted that concentration degree obtainer 110 may use any method of measuring a line of sight and a facial orientation. For example, concentration degree obtainer 110 may include a sensor that detects an eyeball potential of user 10, and may detect at least one of a line of sight or a facial orientation, based on the eyeball potential detected.

Drowsiness degree obtainer 120 detects a degree of drowsiness indicating a drowsiness level of user 10. For example, drowsiness degree obtainer 120 obtains a degree of drowsiness of user 10 from a moving image including user 10 captured by image capturer 210 connected to drowsiness degree obtainer 120. In the present embodiment, drowsiness degree obtainer 120 obtains the degree of drowsiness of user 10 by calculating the degree of drowsiness, based on blinking by user 10 included in an image obtained from image capturer 210.

Here, blinking that drowsiness degree obtainer 120 uses to calculate a degree of drowsiness is, for example, a time required for one blink, the number of blinks within a predetermined time, or a degree of opening of blinks (a degree of opening of eyes).

For example, when a cycle of blinking by user 10 is stable, drowsiness degree obtainer 120 determines that a degree of drowsiness is low, for example, 1, that is, user 10 is not drowsy. Moreover, for example, when user 10 is blinking slowly or a cycle of blinking is short and user 10 is blinking frequently, drowsiness degree obtainer 120 determines that a degree of drowsiness is high, for example, 10, that is, user 10 is drowsy. To put it another way, when the cycle of blinking by user 10 is stable, user 10 is determined to not be drowsy; and when user 10 is blinking slowly and frequently, user 10 is determined to be drowsy. Alternatively, when user 10 takes a long time to blink, that is, user 10 is dozing off, drowsiness degree obtainer 120 may determine a degree of drowsiness to be high; when user 10 takes a short time to blink, drowsiness degree obtainer 120 may determine that a degree of drowsiness to be low. Moreover, when a degree of opening of blinks (a degree of opening of eyes) is small, drowsiness degree obtainer 120 may determine a degree of drowsiness to be high. Furthermore, drowsiness degree obtainer 120 may determine a degree of drowsiness of user 10, based on a combination of indicators related to blinking such as a time required for blinking, the frequency of blinking, and a degree of opening of eyes.

As stated above, drowsiness degree obtainer 120 detects a degree of drowsiness of user 10 by analyzing a moving image including user 10 obtained from image capturer 210. A reference value etc. for a cycle of blinking that drowsiness degree obtainer 120 uses to determine a degree of drowsiness of user 10 may be arbitrarily determined in advance.

It should be noted that a degree of drowsiness is an indicator of how drowsy user 10 is. A degree of drowsiness is represented by a value in a predetermined range such as 0 to 1, 0 to 10, or 0 to 100. For example, a higher degree of drowsiness indicates that user 10 is drowsier, and a lower degree of drowsiness indicates that user 10 is less sleepy, that is, more wakeful.

It should be noted that a value range for a degree of drowsiness is not particularly limited.

Corrector 130 corrects the degree of concentration of user 10, based on the degree of concentration obtained by concentration degree obtainer 110 and the degree of drowsiness obtained by drowsiness degree obtainer 120, to calculate a corrected degree of concentration indicating the degree of concentration corrected. In the present embodiment, corrector 130 corrects the degree of concentration to a predetermined value, based on the degree of drowsiness. A predetermined value is, for example, a value for a degree of drowsiness.

For example, corrector 130 makes the correction so that the degree of concentration of user 10 decreases with an increase in the degree of drowsiness of user 10. For example, corrector 130 corrects the degree of concentration to a smaller value when the degree of drowsiness is a higher value, that is, user 10 is drowsier.

Moreover, corrector 130 makes the correction so that the corrected degree of concentration of user 10 is lower than the degree of concentration of user 10, based on the degree of drowsiness of user 10. In other words, corrector 130 corrects the corrected degree of concentration calculated based on the degree of drowsiness obtained by drowsiness degree obtainer 120 to a value lower than the degree of concentration obtained by concentration degree obtainer 110.

Furthermore, for example, corrector 130 may change a manner of correcting the degree of concentration, based on a position of the predetermined target object such as task 21 or 22. For example, a degree of drowsiness is calculated from a state of blinking by user 10. Accordingly, for example, a movement of an eyelid captured by image capturer 210 differs depending on a positional relationship among a position of user 10, a position of image capturer 210, and a position of task 21 or 22. For this reason, corrector 130 changes a manner of correcting a degree of concentration, based on the position of user 10, the position of image capturer 210, and the position of the predetermined target object such as the position of task 21 or 22. For example, corrector 130 changes, based on the position of the predetermined target object, a correction value for use in calculating a corrected degree of concentration.

For example, storage 160 stores a position of a task in advance. For example, it is assumed that the position of task 21 and the position of task 22 are stored in storage 160 in advance. As shown by FIG. 1, when image capturer 210 is disposed in display device 300, user 10 faces more downward when user 10 is looking at task 22 than when user 10 is looking at task 21. Accordingly, even though user 10 makes the same blinking between when looking at task 21 and when looking at task 22, an amount of change in a degree of opening and closing of blinks (i.e., an amount of change between an eye closure state and an eye open state) is reduced more when user 10 is looking at task 22 than when user 10 is looking at task 21. This causes the blinks to be measured as a slow movement. Consequently, corrector 130 is likely to calculate a higher degree of drowsiness when user 10 is looking at task 22 than when user 10 is looking at task 21. For this reason, corrector 130 calculates a lower degree of drowsiness when user 10 is looking at task 22 than when user 10 is looking at task 21, to reduce a variation in calculation results due to the positions of the tasks. For example, corrector 130 changes values of k and/or c in Equation (1) and Equation (2) described below, based on a position of a predetermined target object displaying a task.

As stated above, for example, corrector 130 changes a correction value based on a degree of drowsiness for use in correcting a degree of concentration, based on a position of a predetermined target object.

It should be noted that when, as with task 22, a task is not located in a direction parallel to an image capturing direction of a camera, a position of a predetermined target object displaying (or describing) the task may be specified by voice in order to measure (calculate) a line of sight direction. In this way, it is possible to reduce a decrease in the accuracy of a degree of drowsiness due to positions of tasks, without changing a manner of correction depending on the positions of the tasks. It should be noted that in this case, concentration degree measurement system 200 may include a sound generator such as an amplifier and a speaker, and the sound generator may be connected to outputter 140.

Moreover, when task 22 is located closer to user 10 than task 21, user 10 looks more downward, and image capturer 210 may not be capable of detecting (capturing) blinking by user 10. In other words, when user 10 is viewed from an image capturer 210 side, there is a possibility that user 10 appears to have closed the eyes. In such a case, for example, corrector 130 may output to outputter 140 information for causing notifier 220 to notify information for inducing user 10 to put image capturer 210 in a position at which blinking by user 10 is detected easily, such as putting task 22 closer to task 21.

Furthermore, corrector 130 may determine a position of a predetermined target object (i.e., a task) by image capturer 210 capturing the predetermined target object.

Moreover, corrector 130 may determine a region of task 21 in accordance with a target object and correct a degree of concentration. For example, in the case where task 21 is a printed form, there is a possibility that user 10 is concentrating when user 10 is performing task 21 looking at texts or equations, etc. on the printed form; and, in contrast, there is a possibility that user 10 is not concentrating when a line of sight of user 10 is focused on outside the printed form. In addition, even when the line of sight of user 10 is directed downward, since a shifting region of the line of sight of user 10 differs depending on a size of a printed form, there may be a case in which user 10 is concentrating and a case in which user 10 is not concentrating. Accordingly, for example, corrector 130 may calculate a size of a printed form by causing image capturer 210 to capture the printed form, and may correct a degree of concentration according to the calculated size of the printed form.

It should be noted that although the present embodiment has described task 22 as a book, the present embodiment is not limited to this example. For example, in the case where a teacher is giving a lecture in front of a desk and user 10 is performing task 21 on the desk, a line of sight of user 10 is directed upward relative to task 21 when user 10 is looking at the teacher. In this case, since the line of sight is directed toward a position different from an image capturing direction, an amount of change in blinks may be reduced even when the same blinking is made. For this reason, corrector 130 may correct a degree of concentration.

Outputter 140 outputs a corrected degree of concentration calculated by corrector 130 correcting a degree of concentration. Outputter 140 is, for example, a communication interface such as a communication adapter communicably connected to notifier 220. Outputter 140 transmits, for example, concentration degree information indicating a corrected degree of concentration to notifier 220. For example, when a corrected degree of concentration exceeds or falls below a predetermined threshold value, outputter 140 transmits concentration degree information. Concentration degree information to be transmitted may be a control signal for controlling notifier 220.

Alternatively, outputter 140 may output, as concentration degree information, voice data or image data to be presented to user 10 or a supervisor of user 10, etc.

Storage 160 is a storage device that stores a control program executed by each of concentration degree obtainer 110, drowsiness degree obtainer 12, and corrector 130. Moreover, storage 160 stores an indicator indicating each of a degree of concentration and a degree of drowsiness, and information such as a threshold value. Storage 160 is realized by, for example, a hard disk drive (HDD) or a flash memory.

Concentration degree measurement device 100 having the above configuration is realized by, for example, computer equipment. Specifically, concentration degree measurement device 100 is realized by a non-volatile memory that stores programs, a volatile memory that is a transitory storage region for executing the programs, an input/output port, a processor that executes the programs, etc. Each function of concentration degree measurement device 100 may be implemented by software executed by a processor, or may be implemented by hardware such as an electrical circuit including at least one electronic component. For example, concentration degree obtainer 110, drowsiness degree obtainer 120, and corrector 130 may be implemented by software executed by a processor, or may be implemented by hardware.

It should be noted that concentration degree measurement device 100 may obtain a degree of concentration and a degree of drowsiness calculated by another device, instead of obtaining an image from image capturer 210. The same holds true for a line of sight and a facial orientation.

Image capturer 210 captures user 10 to generate an image including the face of user 10. Although image capturer 210 is an image sensor sensitive to a visible light band, image capturer 210 may be an infrared image sensor or a thermal image sensor. An image is, for example, a moving image, but may be a still image.

In the present embodiment, image capturer 210 is disposed in display device 300 that displays task 21. Moreover, in the present embodiment, the image capturing direction of image capturer 210 is substantially parallel to the line of sight direction (arrow 11) of user 10 looking at task 21.

Notifier 220 is a device that obtains concentration degree information outputted by outputter 140 and notifies the degree of concentration of user 10, based on the obtained concentration degree information. Notifier 220 notifies user 10 of a corrected degree of concentration by image, voice, etc. Notifier 220 is, for example, a speaker or a display such as display device 300.

Image capturer 210 may have an image capturing function, and may be, for example, a Web camera attached to a personal computer (PC), a camera attached to a tablet terminal, a camera attached to a smartphone, or a camera attached to a television set.

Image capturer 210 captures the face of user 10 to obtain a facial orientation, a line of sight, a body orientation, blinking, a facial expression, etc. of user 10.

It should be noted that in the present embodiment, task 21 user 10 is performing is displayed on display device 300, and task 22 is written in book 310. Examples of display device 300 include a PC, a tablet terminal, a smartphone, and a television. Examples of book 310 include a textbook and a notebook.

### [Concentration Degree Measurement Method]

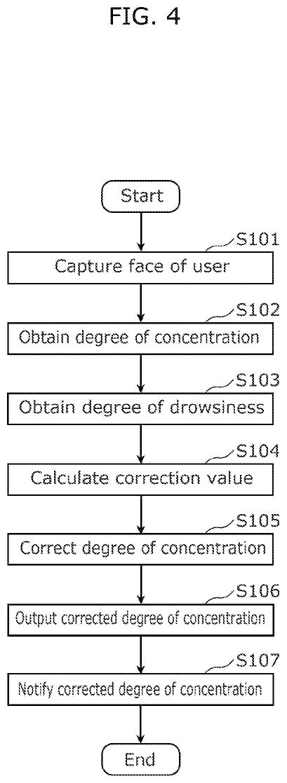

The following describes a concentration degree measurement method performed by concentration degree measurement device 100 according to Embodiment 1, with reference to FIG. 4.

FIG. 4 is a flow chart illustrating a process performed by concentration degree measurement device 100 according to Embodiment 1. It should be noted that the flow chart shown by FIG. 4 shows a process included in concentration degree measurement system 200, such as an operation of image capturer 210.

First, image capturer 210 captures the face of user 10 (step S101). A timing, a cycle, etc. at which image capturer 210 captures the face of user 10 is not particularly limited. For example, image capturer 210 starts capturing at a timing when user 10 activates concentration degree measurement device 100.

Next, concentration degree obtainer 110 obtains a degree of concentration of user 10 (step S102). In the present embodiment, first, concentration degree obtainer 110 obtains images showing the face of user 10 captured at different times by image capturer 210 in step S101. Next, concentration degree obtainer 110 calculates an amount of change in a line of sight and a facial orientation of user 10 from the obtained images. Subsequently, concentration degree obtainer 110 obtains a degree of concentration of user 10 by calculating the degree of concentration of user 10, based on the calculated amount of change and a concentration degree information table indicating a predetermined correspondence relationship between amounts of change and degrees of concentration.

Then, drowsiness degree obtainer 120 obtains a degree of drowsiness of user 10 (step S103). In the present embodiment, first, drowsiness degree obtainer 120 obtains images showing the face of user 10 captured at different times by image capturer 210 in step S101. Next, drowsiness degree obtainer 120 calculates a drowsiness feature amount such as the number of times user 10 blinks within a predetermined time or a time required for each blink, from the obtained images. Subsequently, drowsiness degree obtainer 120 obtains a degree of drowsiness of user 10 by calculating the degree of drowsiness of user 10, based on the calculated drowsiness feature amount and a drowsiness degree information table indicating a predetermined correspondence relationship between drowsiness feature amounts and degrees of drowsiness.

After that, corrector 130 calculates a correction value, based on the degree of drowsiness of user 10 obtained by drowsiness degree obtainer 120 (step S104).

Next, corrector 130 corrects the degree of concentration of user 10 obtained by concentration degree obtainer 110 with the correction value (step S105). In step S104 and step S105, for example, corrector 130 calculates a correction value by multiplying a degree of drowsiness by a predetermined coefficient, and calculates a difference between the correction value and a degree of concentration to obtain a corrected degree of concentration that is a value obtained by correcting the degree of concentration. For example, it is assumed that a degree of concentration is “5,” a degree of drowsiness is “2,” and a coefficient is 0.5. In this case, corrector 130 calculates a corrected degree of concentration as 5−2×0.5=4. Needless to say, a coefficient may be arbitrarily determined, need not be a constant number, and may be, for example, a value that changes in accordance with a degree of drowsiness.

For example, a degree of concentration may be represented by any numerical level as a digital value (an integer). For example, a degree of concentration may be represented by a 10-point scale ranging from 1 to 10.

Moreover, for example, a degree of drowsiness may be represented by any numerical level as a digital value. For example, a degree of drowsiness may be represented by a 10-point scale ranging from 1 to 10.

When a level representing a degree of concentration is A and a level representing a degree of drowsiness is B, A′ that is a level representing a corrected degree of concentration may be calculated as indicated by Equation (1), Equation (2), and Equation (3).

A′=round[A−k×(B−c)] if B>c Equation (1)

A′=round[A−k×B] if B≤c Equation (2)

A′=0 if A′<0 Equation (3)

It should be noted that k and c are positive integers that are arbitrarily determined in advance. Moreover, round is a function for outputting a digital value.

For example, when k is 1, c is 4, A is 7, and B is 5, A′ is 6. In this case, for example, k×(B−c)=1 is the correction value based on the degree of drowsiness, and the corrected degree of concentration is calculated based on a difference between the degree of concentration and the correction value.

It should be noted that when a degree of drowsiness exceeds a predetermined value, a corrected degree of concentration may be obtained by calculating a difference between a predetermined correction value and a degree of concentration.

Moreover, a degree of concentration, a corrected degree of concentration, and a degree of drowsiness may be represented by a real number value within a predetermine range (e.g., 0 to 1) as an analog value (a real number).

Furthermore, the method of calculating a corrected degree of concentration may be changed based on the details of the task user 10 is performing, the environment in which user 10 is located such as temperature and humidity, and an attribute such as the sex, age, etc. of user 10. For example, a value of k is set higher with an increase in impact of a degree of drowsiness on a corrected degree of concentration.

Moreover, for example, user information indicating how a degree of concentration has changed in the past based on the environment of user 10, a time, and working hours may be obtained in advance, and the method of calculating a corrected degree of concentration may be determined based on the user information.

Furthermore, for example, when a current environment induces user 10 to be drowsy, values of k and/or c may be changed so that a correction value becomes higher.

Moreover, for example, all-user information indicating how degrees of concentration have changed in the past based on the environments of users other than user 10 using concentration degree measurement device 100, times, and working hours may be obtained in advance, and the method of calculating a corrected degree of concentration may be determined based on the all-user information.

Furthermore, for example, corrector 130 may change a correction value based on a degree of drowsiness for use in correcting a degree of concentration, based on a position of a predetermined target object (e.g., a display device that displays task 21, book 310 in which task 22 is written). For example, corrector 130 may determine at least one of values of k or c, based on the position of the predetermined target object.

Then, outputter 140 outputs the corrected degree of concentration (step S106). In step S106, for example, outputter 140 outputs to notifier 220 image information, voice information, etc. having a numerical value indicating a corrected degree of concentration. Outputter 140 may output information suitable for a configuration of notifier 220. For example, when notifier 220 is a display device such as a display, outputter 140 outputs image information. In addition, for example, when notifier 220 is an audio device, outputter 140 outputs voice information. It should be noted that when notifier 220 is capable of generating an image, voice, etc. using only a numerical value of a corrected degree of concentration, outputter 140 may output information including the numerical value of the corrected degree of concentration.

Finally, notifier 220 notifiers user 10 of the obtained corrected degree of concentration (step S107). In step S107, for example, when notifier 220 is a display device such as a display, notifier 220 outputs an image indicating a corrected degree of concentration. In addition, for example, when notifier 220 is an audio device, notifier 220 outputs voice indicating a corrected degree of concentration.

For example, outputter 140 may output information indicating a corrected degree of concentration when a degree of concentration is higher than a predetermined threshold value. Alternatively, outputter 140 may output information indicating a corrected degree of concentration when a degree of concentration is lower than a predetermined threshold value. Moreover, when there is such a condition for outputting by outputter 140, for example, user 10 may determine arbitrarily whether to cause outputter 140 to output the information indicating the corrected degree of concentration when the degree of concentration is higher than the predetermined threshold value or to cause outputter 140 to output the information indicating the corrected degree of concentration when the degree of concentration is lower than the predetermined threshold value. In this case, for example, concentration degree measurement system 200 may include an operation receiver such as a touch panel for receiving an instruction from user 10.

For example, in the case where outputter 140 outputs information indicating a corrected degree of concentration when a degree of concentration is higher than a predetermined threshold value, corrector 130 may cause outputter 140 to output information that gives a message encouraging user 10 to maintain motivation, for example, information causing notifier 220 to display a message such as “Your degree of concentration is high” or “You are keeping a high degree of concentration.” In this way, it is possible to induce user 10 to concentrate on a task. Moreover, when a degree of concentration of user 10 is high, it is possible to avoid disturbing the concentration by outputter 140 outputting the information indicating the corrected degree of concentration when the degree of concentration is lower than the predetermined threshold value; and when a degree of concentration of user 10 is low, it is possible to induce user 10 to concentrate by sending a notice (a notification). For example, it is possible to induce user 10 to improve a degree of concentration by notifying user 10 of a message such as “Your degree of concentration is decreasing” or a value of the degree of concentration. Furthermore, corrector 130 may change information that outputter 140 is caused to output (i.e., information that notifier 220 is caused to notify) between when the degree of concentration (a corrected degree of concentration) of user 10 is high and when the degree of concentration of user 10 is low. For example, when a degree of concentration is higher than a predetermined threshold value, corrector 130 may cause outputter 140 to output only a value of the degree of concentration; and when a degree of concentration is lower than the predetermined threshold value, corrector 130 may cause notifier 220 to induce user 10 to improve the degree of concentration, by causing outputter 140 to output information including an encouraging message together with the value.

It should be noted that a predetermined threshold value may be determined arbitrarily in advance, and is not particularly limited.

### [Advantageous Effects Etc.]

As described above, concentration degree measurement device 100 according to Embodiment 1 includes: concentration degree obtainer 110 that obtains a degree of concentration of user 10; drowsiness degree obtainer 120 that obtains a degree of drowsiness of user 10; corrector 130 that corrects the degree of concentration, based on the degree of drowsiness; and outputter 140 that outputs a corrected degree of concentration indicating the degree of concentration corrected by corrector 130.

With such a configuration, corrector 130 is capable of correcting a degree of concentration of user 10, based on a degree of drowsiness of user 10. For this reason, calculating the degree of concentration to be high is suppressed even though the degree of drowsiness is high and the degree of concentration is low. As stated above, concentration degree measurement device 100 according to the present disclosure is capable of measuring a degree of concentration with high accuracy.

Moreover, for example, corrector 130 makes the correction so that the degree of concentration decreases with an increase in the degree of drowsiness.

A high degree of drowsiness of user 10 can be considered equivalent to a low degree of concentration of user 10. Accordingly, by correcting a degree of concentration of user 10 to be lower as a degree of drowsiness of user 10 is higher, the degree of concentration is measured with high accuracy.

Moreover, for example, corrector 130 makes the correction so that the corrected degree of concentration is lower than the degree of concentration, based on the degree of drowsiness.

A degree of concentration of user 10 is considered to decrease with an increase in a degree of drowsiness of user 10. For this reason, by correcting the degree concentration to be lower based on the degree of drowsiness, the degree of concentration is measured with ease and high accuracy.

Moreover, for example, concentration degree obtainer 110 obtains the degree of concentration by calculating the degree of concentration, based on at least one of a line of sight or a facial orientation of user 10 included in an image obtained from image capturer 210. Furthermore, for example, drowsiness degree obtainer 120 obtains the degree of drowsiness by calculating the degree of drowsiness, based on blinking by the person in the image.

Such a configuration makes it possible to obtain a degree of drowsiness and a degree of concentration of user 10 from the same image. Accordingly, the degree of concentration and the degree of drowsiness are calculated using the simple configuration.

Moreover, for example, concentration degree obtainer 110 obtains the degree of concentration by calculating, from images obtained from image capturer 210 and including user 10 and a predetermined target object (e.g., display device 300 that displays task 21, book 310 in which task 22 is written), an amount of temporal change in at least one of the line of sight or the facial orientation of user 10 toward the predetermined target object.

For example, when user 10 is performing task 21 such as learning materials while looking at display device 300, a predetermined targe object, a degree of concentration of user 10 is determined to be higher as a time for which user 10 is looking at display device 300 is longer. For this reason, with such a configuration, the degree of concentration is measured with higher accuracy.

Moreover, for example, corrector 130 may change a correction value based on the degree of drowsiness for use in correcting the degree of concentration, based on a position of a predetermined target object (e.g., display device 300 that displays task 21, book 310 in which task 22 is written).

For example, a degree of drowsiness of user 10 is calculated based on a time required for one blinking by user 10. Accordingly, for example, when user 10 is performing task 21 such as learning materials while looking at display device 300, a predetermined target object, an amount of opening and closing by the eyelids captured by image capturer 210 changes depending on a positional relationship between display device 300, image capturer 210, and user 10. Therefore, such a configuration reduces deterioration of accuracy of measuring the degree of concentration due to the positional relationship between the predetermined target object, image capturer 210, and user 10.

A concentration degree measurement method according to the present disclosure includes: obtaining a degree of concentration of user 10; obtaining a degree of drowsiness of user 10; correcting the degree of concentration, based on the degree of drowsiness; and outputting a corrected degree of concentration indicating the degree of concentration corrected in the correcting.

Such a method makes it possible to correct a degree of concentration of user 10, based on a degree of drowsiness of user 10. For this reason, calculating the degree of concentration to be high is suppressed even though the degree of drowsiness is high and the degree of concentration is low. As stated above, the concentration degree measurement method according to the present disclosure makes it possible to measure a degree of concentration with high accuracy.

Furthermore, one aspect of the present disclosure can be realized as a program causing a computer to execute the above concentration degree measurement method. Alternatively, it can be realized as a computer-readable recording medium having the program recorded thereon.

It should be noted that the present disclosure may be implemented as a program causing a computer to execute the steps included in the above concentration degree measurement method. Moreover, the present disclosure may be implemented as a non-transitory computer-readable recording medium, such as CD-ROM, with the program recorded thereon. Furthermore, the present disclosure may be implemented as information, data, or signal indicating the program. The program, information, data, and signal may be distributed via a communication network such as the Internet.

### Embodiment 2

Next, the following describes a concentration degree measurement device according to Embodiment 2. It should be noted that the description of the concentration degree measurement device according to Embodiment 2 focuses mainly on differences from the concentration degree measurement device according to Embodiment 1, substantially same constituent elements are assigned the same reference signs, and description thereof may be partially simplified or omitted.

### [Configuration]

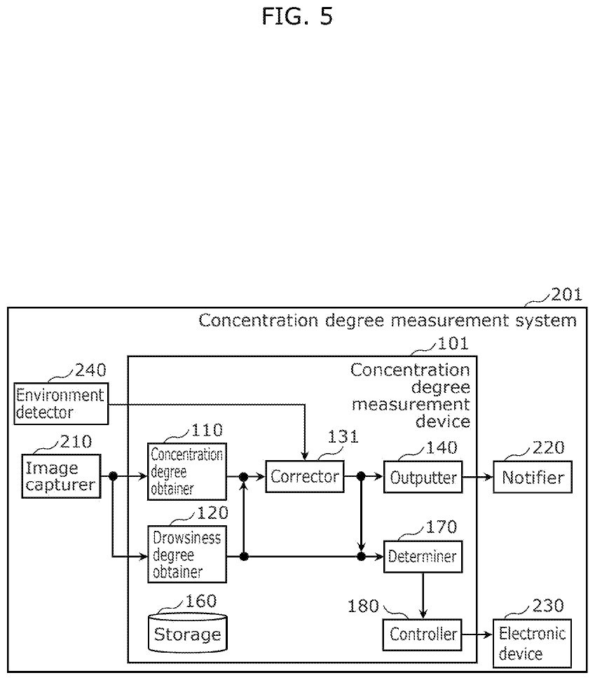

The following describes a specific configuration of the concentration degree measurement device according to Embodiment 2 with reference to FIG. 5.

FIG. 5 is a block diagram illustrating a characteristic functional configuration of concentration degree measurement device 101 according to Embodiment 2. It should be noted that FIG. 5 shows a characteristic functional configuration of concentration degree measurement system 201 including a peripheral configuration of concentration degree measurement device 101.

Concentration degree measurement system 201 causes concentration degree measurement device 101 to measure a degree of concentration of a person (e.g., user 10 shown by FIG. 1) using an image captured by image capturer 210 and to notify the person of the degree of concentration. Concentration degree measurement system 201 includes image capturer 210, concentration degree measurement device 101, notifier 220, electronic device 230, and environment detector 240.

Concentration degree measurement device 101 measures a degree of concentration of user 10. Concentration degree measurement device 101 includes concentration degree obtainer 110, drowsiness degree obtainer 120, corrector 131, outputter 140, storage 160, determiner 170, and controller 180.

Corrector 131 corrects the degree of concentration (a provisional degree of concentration) of user 10, based on the degree of concentration obtained by concentration degree obtainer 110 and the degree of drowsiness obtained by drowsiness degree obtainer 120, to calculate a corrected degree of concentration indicating the degree of concentration corrected. In the present embodiment, corrector 131 corrects the provisional degree of concentration to a predetermined value, based on the degree of drowsiness. A predetermined value is, for example, a value for a degree of drowsiness.

Moreover, corrector 131 changes a manner of correcting the degree of concentration of user 10, based on environmental information indicating an environmental state of user 10. For example, corrector 131 obtains environmental information from environment detector 240 that detects, as the environmental information, information about temperature, humidity, brightness, environmental sound, etc. of the environment in which user 10 is located.

Environments make a difference as to whether user 10 is likely to concentrate, become drowsy, etc. For this reason, for example, when an environment does not easily induce user 10 to become very drowsy, a low degree of drowsiness is likely to be calculated since a degree of drowsiness of user 10 is unlikely to be high, and accuracy is decreased. Accordingly, corrector 131 changes a correction manner depending on the environment.

Determiner 170 determines a factor for the corrected degree of concentration of user 10, based on the degree of drowsiness and corrected degree of concentration of user 10. For example, determiner 170 determines whether the corrected degree of concentration of user 10 is lower than a first threshold value, and when determiner 170 determines that the corrected degree of concentration of user 10 is lower than the first threshold value, determiner 170 determines a factor that has caused the corrected degree of concentration of user 10 to be the degree of concentration lower than the first threshold value, based on the degree of drowsiness and corrected degree of concentration of user 10.

For example, examples of a factor that causes a low degree of concentration of user 10 may include an external factor such as a television and an internal factor such as drowsiness. When a degree of concentration of user 10 is low, determiner 170 determines how much drowsiness has influenced a factor causing the low degree of concentration.

For example, determiner 170 determines whether a proportion of drowsiness to the factor that has caused the corrected degree of concentration of user 10 to be lower than the first threshold value is higher than a second threshold value.

It should be noted that the first threshold value and the second threshold value may be arbitrarily determined in advance, and are not particularly limited. For example, the first threshold value and the second threshold value are stored in storage 160.

Determiner 170 outputs a result of the determination to controller 180.

Controller 180 controls electronic device 230 that induces user 10 to wake up or concentrate, based on the degree of drowsiness and corrected degree of drowsiness of user 10. Specifically, controller 180 controls electronic device 230, based on the result of the determination obtained from determiner 170.

For example, when determiner 170 determines that the proportion of the drowsiness in the factor that has caused the corrected degree of concentration of user 10 to be lower than the first threshold value is higher than the second threshold value, controller 180 causes electronic device 230 to induce user 10 to wake up. On the other hand, for example, when determiner 170 determines that the proportion of the drowsiness in the factor that has caused the corrected degree of concentration of user 10 to be lower than the first threshold value is not higher than the second threshold value, controller 180 causes electronic device 230 to induce user 10 to concentrate. As stated above, when the factor that has caused the low degree of concentration of user 10 is drowsiness, controller 180 causes electronic device 230 to reduce the drowsiness of user 10, that is, to induce user 10 to wake up.

In contrast, when the factor that has caused the low degree of concentration of user 10 is not drowsiness, controller 180 causes electronic device 230 to induce user 10 to concentrate. For example, when a degree of concentration is low regardless of drowsiness, as shown by FIG. 2B, there is a case in which user 10 gets distracted instead of focusing on task 21 or 22. In such a case, changing an environment to an environment in which user 10 is likely to feel calm improve the degree of concentration of user 10.

For example, an environment may be changed to an opposite state between when user 10 is induced to wake up and when user 10 is induced to concentrate. User 10 is induced to concentrate by increasing an environmental temperature, and is induced to wake up by decreasing an environmental temperature. As stated above, by inversely controlling an environmental condition for user 10, it is possible to induce the wake-up and concentration of user 10 separately. In view of this, for example, controller 180 performs inverse control on electronic device 230 between when user 10 is induced to wake up and when user 10 is induced to concentrate. Accordingly it is possible to appropriately induce user 10 to wake up or concentrate.

It should be noted that in order to induce concentration or wakefulness, electronic device 230 may perform outputting music, setting a screen background color of display device 300, giving voice instructions, etc. that produce a conflicting effect. For example, electronic device 230 may play stimulative music to induce user 10 to wake up, or may play relaxing music to induce user 10 to concentrate. Moreover, for example, electronic device 230 may change the screen back ground color of display device 300 to a warm color (e.g., red) to induce user 10 to wake up, or may change the screen back ground color to a cold color (e.g., blue) to induce user 10 to concentrate. Furthermore, for example, electronic device 230 may set a comfortable environmental temperature to induce user 10 to wake up, or may set an uncomfortable environmental temperature (e.g., an excessively high temperature or an excessively low temperature) to induce user 10 to concentrate. In addition, for example, electronic device 230 may decrease a degree of difficulty of task 21 to induce user 10 to wake up, or may increase a degree of difficulty of task 21 to induce user 10 to concentrate.

Attribute information indicating preferences of user 10 for music, temperature, etc. may be stored in storage 160 in advance. For example, controller 180 may cause electronic device 230 to operate to induce user 10 to wake up or concentrate, based on the attribute information.

It should be noted that R that indicates a proportion of drowsiness in a factor calculated by determiner 170 may be calculated by, for example, Equation (4) below.

R=B/A′ Equation (4)

It should be noted that R may be a value in proportion to the magnitude of a correction value. For example, R may be calculated by Equation (5) below.

R=B/(A−A′) Equation (5)

Concentration degree measurement device 101 having the above configuration is realized by, for example, computer equipment. Specifically, concentration degree measurement device 101 is realized by a non-volatile memory that stores programs, a volatile memory that is a transitory storage region for executing the programs, an input/output port, a processor that executes the programs, etc. Each function of concentration degree measurement device 101 may be implemented by software executed by a processor, or may be implemented by hardware such as an electrical circuit including at least one electronic component. For example, concentration degree obtainer 110, drowsiness degree obtainer 120, corrector 131, determiner 170, and controller 180 may be implemented by software executed by a processor, or may be implemented by hardware.

Electronic device 230 is for inducing user 10 to wake up or concentrate. Examples of electronic device 230 include air-conditioning equipment that controls temperature, humidity, etc., audio equipment, and lighting equipment. The number of electronic devices 230 used for concentration degree measurement system 201 may be one or more.

Moreover, for example, electronic device 230 may be an electronic device that performs inverse control to switch between inducing user 10 to wake up and inducing user 10 to concentrate. For example, when electronic device 230 is air-conditioning equipment, electronic device 230 increases an environmental temperature of user 10 to induce user 10 to concentrate, and decreases the environmental temperature of user 10 to induce user 10 to wake up.

Environment detector 240 is a sensor that detects an environment of user 10. Examples of environment detector 240 include a temperature sensor that detects a temperature, a humidity sensor that detects a humidity, a sound detector that detects a sound volume, and a light sensor that detects a brightness.

### [Concentration Degree Measurement Method]

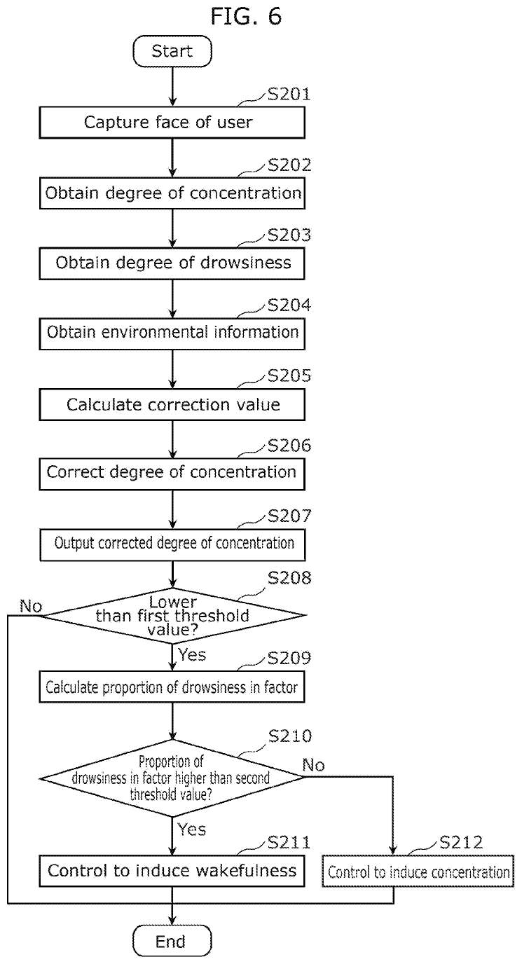

Next, the following describes a concentration degree measurement method performed by concentration degree measurement device 101 according to Embodiment 2 with reference to FIG. 6.

FIG. 6 is a flow chart illustrating a process performed by concentration degree measurement device 101 according to Embodiment 2. It should be noted that the flow chart shown by FIG. 6 illustrates the process included in concentration degree measurement system 201, such as operations of image capturer 210.

First, image capturer 210 captures the face of user 10 (step S201).

Next, concentration degree obtainer 110 obtains a degree of concentration of user 10 (step S202).

Then, drowsiness degree obtainer 120 obtains a degree of drowsiness of user 10 (step S203).

Steps S201 to S203 are the same as steps S101 to S103 shown by FIG. 4.

After that, corrector 131 obtains environmental information from environment detector 240 (step S204).

Next, corrector 131 calculates a correction value, based on the degree of drowsiness of user 10 obtained by drowsiness degree obtainer 120 and the environmental information obtained from environment detector 240 (step S205).

Then, corrector 130 corrects the degree of concentration of user 10 obtained by concentration degree obtainer 110 with the correction value (step S206).

In step S205 and step S206, for example, corrector 131 calculates a correction value by multiplying a degree of drowsiness by a predetermined coefficient, and calculates a difference between the correction value and a degree of concentration to obtain a corrected degree of concentration that is a value obtained by correcting the degree of concentration. Here, for example, the coefficient is changed based on environmental information. For example, when a temperature detected as the environmental information detected by environment detector 240 is within a predetermined range, a coefficient (e.g., k in Equation (1)) is set to “2”; and in other cases, the coefficient is set to “1.” As stated above, for example, corrector 131 changes a correction value, based on environmental information.

After that, outputter 140 outputs the corrected degree of concentration (step S207). In step S207, for example, outputter 140 outputs to notifier 220 image information, voice information, etc. having a numerical value indicating a corrected degree of concentration.

It should be noted that, though not shown here, for example, notifier 220 notifies user 10 of an obtained corrected degree of concentration after step S207.

Next, determiner 170 obtains the corrected degree of concentration of user 10 calculated by corrector 131 and determines whether the obtained corrected degree of concentration is lower than a first threshold value (step S208).

When determiner 170 determines that the corrected degree of concentration is not lower than the first threshold value (No in step S208), for example, determiner 170 completes the process, regarding the degree of concentration of user 10 as high. Concentration degree measurement system 201 may start again the process from step S201. Outputter 140 may output to notifier 220 information indicating that the the degree of concentration of user 10 is high.

In contrast, when determiner 170 determines that the corrected degree of concentration is lower than the first threshold value (Yes in step S208), determiner 170 determines a factor that has caused the corrected degree of concentration to be lower than the first threshold value, based on the degree of drowsiness and corrected degree of concentration of user 10 (step S209). Specifically, first, in step S209, for example, determiner 170 obtains the degree of drowsiness of user 10 from drowsiness degree obtainer 120 and calculates a proportion of drowsiness in a whole factor that has caused the degree of concentration of user 10 to be low, based on the obtained degree of drowsiness of user 10 and the corrected degree of concentration of user 10 obtained in step S208. For example, determiner 170 calculates the proportion of the drowsiness in the factor using Equation (4) or Equation (5).

Then, determiner 170 determines whether the proportion of the drowsiness in the factor calculated in step S209 is higher than a second threshold value (step S210).

When determiner 170 determines that the proportion of the drowsiness in the factor is higher than the second threshold value (Yes in step S210), controller 180 causes electronic device 230 to induce user 10 to wake up (step S211).

On the other hand, when determiner 170 determines that the proportion of the drowsiness in the factor is not higher than the second threshold value (No in step S210), controller 180 causes electronic device 230 to induce user 10 to concentrate (step S212).

### [Advantageous Effects Etc.]

As described above, concentration degree measurement device 101 according to Embodiment 2 includes: concentration degree obtainer 110 that obtains a degree of concentration of user 10; drowsiness degree obtainer 120 that obtains a degree of drowsiness of user 10; corrector 131 that corrects the degree of concentration, based on the degree of drowsiness; and outputter 140 that outputs a corrected degree of concentration indicating the degree of concentration corrected by corrector 131. Concentration degree measurement device 101 further includes controller 180 that controls electronic device 230 that induces user 10 to wake up or concentrate, based on the degree of drowsiness and corrected degree of drowsiness of user 10.

With such a configuration, since controller 180 controls electronic device 230, based on a degree of concentration (a corrected degree of concentration) of user 10 corrected to an appropriate value by corrector 131, controller 180 is capable of inducing user 10 to wake up or concentrate at an appropriate timing.

Moreover, for example, concentration degree measurement device 101 further includes determiner 170 that determines whether the corrected degree of concentration of user 10 is lower than a first threshold value, and determines, when determiner 170 determines that the corrected degree of concentration of user 10 is lower than the first threshold value, a factor that has caused the corrected degree of concentration of user 10 to be the degree of concentration lower than the first threshold value, based on the degree of drowsiness and corrected degree of concentration of user 10. In this case, controller 180 controls electronic device 230, based on the result of the determination by determiner 170.

With such a configuration, since controller 180 controls electronic device 230, based on a result of determination by determiner 170, controller 180 reduces unnecessary urging of user 10 to wake up or concentrate.

Moreover, for example, when determiner 170 determines that a proportion of drowsiness in a factor is higher than a second threshold value, controller 180 causes electronic device 230 to induce user 10 to wake up; and when determiner 170 determines that the proportion of the drowsiness in the factor is not higher than the second threshold value, controller 180 causes electronic device 230 to induce user 10 to concentrate, the factor having caused the corrected degree of concentration to be lower than the first threshold value.

With such a configuration, since determiner 170 appropriately determines whether to urge user 10 to wake up or concentrate, controller 180 is capable of further appropriately urging user 10 to wake up or concentrate.

Moreover, for example, controller 180 performs inverse control on electronic device 230 between when user 10 is induced to wake up and when user 10 is induced to concentrate.

For example, an increase in environmental temperature induces user 10 to concentrate. In contrast, a decrease in environmental temperature induces user 10 to wake up. Furthermore, for example, an increase in environmental noise (environmental sound volume) induces user 10 to wake up. In contrast, a decrease in environmental noise induces user 10 to concentrate. As stated above, by switching control modes, it is possible to cause one electronic device 230 such as air-conditioning equipment and audio equipment to induce user 10 to wake up or concentrate. Accordingly, by controller 180 performing inverse control on electronic device 230 between when electronic device 230 induces user 10 to wake up and when electronic device 230 induces user 10 to concentrate, it is possible to induce user 10 to wake up or concentrate using the simple configuration.

Moreover, for example, corrector 131 changes a manner of correcting the degree of concentration, based on environmental information indicating an environmental state of user 10.

For example, when an environmental temperature is relatively high, a change in a degree of concentration and a degree of drowsiness of user 10 has a tendency, such as a tendency of the degree of concentration and the degree of drowsiness to increase, depending on an environmental condition of the person. For this reason, by correcting the degree of concentration, based on the environmental condition of user 10, concentration degree measurement device 101 according to the present disclosure is capable of measuring the degree of concentration with higher accuracy.

### OTHER EMBODIMENTS

The concentration degree measurement devices etc. according to one or more aspects have been described above based on the embodiments; however, the present disclosure is not limited to these embodiments. A scope of the present disclosure includes forms obtained by making various modifications conceived by a person skilled in the art to the embodiments, and forms obtained by combining constituent elements in different embodiments, as long as the forms do not depart from the essence of the present disclosure.

For example, step S102 and step S103 shown by FIG. 4 may be performed in reverse order or at the same time. In this manner, the flow charts according to the present disclosure are merely examples and the steps may be arbitrarily switched within the scope of the claims.

Moreover, a method of communication between devices described in the aforementioned embodiments is not particularly limited. When the devices communicate wirelessly with each other, the method used for wireless communication (communication standard) is near-field communication such as Zigbee (registered trademark), Bluetooth (registered trademark), or a wireless local area network (LAN). Alternatively, the method used for wireless communication (communication standard) may be communication via a wide area communication network such as the Internet. In addition, the devices may perform wired communication, instead of wireless communication. Specifically, the wired communication includes power line communication, or communication using a wired LAN.

Furthermore, for example, concentration degree measurement devices 100 and 101 may transmit information in which a task such as e-learning that is a learning style using the Internet, a response state of user 10, a degree of drowsiness, and a degree of concentration (corrected degree of concentration) are associated, to a server used by a manager providing a task. In this manner, the manager can change a current task to a task having content that makes user 10 less drowsy and increases a degree of concentration, based on the information received by the server. In addition, the manager can assess how much user 10 can complete the task, based on the information received by the server.

Moreover, for example, information in which a task, a response state of user 10, a degree of drowsiness, and a degree of concentration are associated may be transmitted to a device such as a PC used by a supervisor (e.g., a parent when user 10 is a student) of user 10. This allows the supervisor to browse the information, and thus the supervisor can determine whether user 10 is performing a task suitable for the competence of user 10.

Furthermore, for example, concentration degree measurement devices 100 and 101 may cause display device 300 etc. to display information in which a task, a response state of user 10, a degree of drowsiness, and a degree of concentration are associated so that user 10 can browse the information in real time. In this way, concentration degree measurement devices 100 and 101 is capable of inducing user 10 to exercise self-control for a degree of concentration by giving an objective indicator to user 10.

Moreover, for example, in the aforementioned embodiments, concentration degree obtainer 120 obtains an amount of change in line of sight and facial orientation within a predetermined time. The predetermined time may be arbitrarily set.

Furthermore, in the aforementioned embodiments, a process performed by a particular processing unit may be performed by other processing units. Moreover, the order of processes may be changed, or the processes may be performed in parallel. In addition, the constituent elements included in concentration degree measurement devices 100 and 101 may be distributed to a plurality of devices. For example, constituent elements included in one device may be included in another device.

For example, the process described in each of the aforementioned embodiments may be realized via integrated processing using a single device (system) or may be realized via decentralized processing using a plurality of devices. In addition, the processor that executes the above program may be a single processor or a plurality of processors. In other words, integrated processing may be performed or decentralized processing may be performed.

Moreover, in the aforementioned embodiments, all or part of the constituent elements of the processor included in concentration degree measurement device 100, such as corrector 130, may be configured using dedicated hardware or may be realized by executing a software program suitable for the respective constituent elements. Each of the constituent elements may be implemented by a program executing element, such as a central processing unit (CPU) or processor, reading and executing a software program recorded on a recording medium such as a hard disk drive (HDD) or a semiconductor memory.

Furthermore, the constituent elements of the processor included in concentration degree measurement device 100, such as corrector 130, may be configured using one or more electronic circuits. The one or more electronic circuits may be each a general-purpose circuit or a dedicated circuit.

The one or more electronic circuits may include, for example, a semiconductor device, an integrated circuit (IC), or a large scale integration (LSI). The IC or LSI may be integrated into a single chip or multiple chips. Due to a difference in the degree of integration, the electronic circuit referred here to as an IC or LSI may be referred to as a system LSI, very large scale integration (VLSI), or ultra large scale integration (ULSI). In addition, a field programmable gate array (FPGA) that is programmable after manufacturing of the LSI can be used for the same purposes.

Moreover, the general and specific aspects of the present disclosure may be implemented using a system, a device, a method, an integrated circuit, or a computer program. Alternatively, these aspects may be implemented using a non-transitory computer-readable recording medium such as an optical disk, HDD, or a semiconductor memory having the computer program recorded thereon. In addition, those aspects may be implemented using any combination of systems, devices, methods, integrated circuits, computer programs, or recording media.

Furthermore, for each of the aforementioned embodiments, various modifications, replacements, additions, omissions, etc. can be carried out within the scope of the claims or equivalents thereof.

### INDUSTRIAL APPLICABILITY

The present disclosure can be used as a concentration degree device capable of measuring a degree of concentration with high accuracy, and can be applied to a device, a method, etc. that assist various types of tasks such as studying or driving.

### REFERENCE SIGNS LIST

10 user
11, 12, 13, 14, 15 arrow
21, 22 task
100, 101 concentration degree measurement device
110 concentration degree obtainer
120 drowsiness degree obtainer
130, 131 corrector
140 outputter
160 storage
170 determiner
180 controller
200, 201 concentration degree measurement system
210 image capturer
220 notifier
230 electronic device
240 environment detector
300 display device
310 book
320 concentration target object

1. A concentration degree measurement device, comprising:
a concentration degree obtainer that obtains a degree of concentration of a person;
a drowsiness degree obtainer that obtains a degree of drowsiness of the person;
a corrector that corrects the degree of concentration, based on the degree of drowsiness;
an outputter that outputs a corrected degree of concentration indicating the degree of concentration corrected by the corrector;
a determiner that determines a factor for the corrected degree of concentration, based on the degree of drowsiness and the corrected degree of concentration; and
a controller that controls an electronic device, based on a result of the determination by the determiner, the electronic device inducing the person to wake up or concentrate.

2. The concentration degree measurement device according to claim 1,
wherein the corrector makes the correction so that the degree of concentration decreases with an increase in the degree of drowsiness.

3. The concentration degree measurement device according to claim 1,
wherein the corrector makes the correction so that the corrected degree of concentration is lower than the degree of concentration, based on the degree of drowsiness.

4. The concentration degree measurement device according to claim 1,
wherein the determiner determines whether the corrected degree of concentration is lower than a first threshold value, and when the determiner determines that the corrected degree of concentration is lower than the first threshold value, the determiner determines a factor that has caused the corrected degree of concentration to be lower than the first threshold value.

5. The concentration degree measurement device according to claim 1,
wherein when the determiner determines that a proportion of drowsiness in the factor is higher than a second threshold value, the controller causes the electronic device to induce the person to wake up; and when the determiner determines that the proportion of the drowsiness in the factor is not higher than the second threshold value, the controller causes the electronic device to induce the person to concentrate.

6. The concentration degree measurement device according to claim 1,
wherein the controller performs inverse control on the electronic device between when the electronic device induces the person to wake up and when the electronic device induces the person to concentrate.

7. The concentration degree measurement device according to claim 1,
wherein the corrector changes a manner of correcting the degree of concentration, based on environmental information indicating an environmental state of the person.

8. The concentration degree measurement device according to claim 1,
wherein the concentration degree obtainer obtains the degree of concentration by calculating the degree of concentration, based on at least one of a line of sight or a facial orientation of the person in an image obtained from an image capturer, and
the drowsiness degree obtainer obtains the degree of drowsiness by calculating the degree of drowsiness, based on blinking by the person in the image.

9. The concentration degree measurement device according to claim 8,
wherein the concentration degree obtainer obtains the degree of concentration by calculating, from images obtained from the image capturer and showing the person and a predetermined target object, an amount of temporal change in the at least one of the line of sight or the facial orientation toward the predetermined target object.

10. The concentration degree measurement device according to claim 9,
wherein the corrector changes a correction value based on the degree of drowsiness, based on a position of the predetermined target object, the correction value being used to correct the degree of concentration.

11. A concentration degree measurement method, comprising:
obtaining a degree of concentration of a person;
obtaining a degree of drowsiness of the person;
correcting the degree of concentration, based on the degree of drowsiness;
outputting a corrected degree of concentration indicating the degree of concentration corrected in the correcting;
determining a factor for the corrected degree of concentration, based on the degree of drowsiness and the corrected degree of concentration; and
controlling an electronic device, based on a result of the determination in the determining, the electronic device inducing the person to wake up or concentrate.

12. A non-transitory computer-readable recording medium for use in a computer, the recording medium having a computer program recorded thereon for causing the computer to execute the concentration degree measurement method according to claim 11.
